(19) United States
(12) Patent Application Publication
(10) Pub. No.: US 2026/0334837 A1
(43) Pub. Date: Oct. 29, 2026

(54) URINE ELECTRICAL CHARACTERISTIC MEASUREMENT DEVICE, URINE ELECTRICAL CHARACTERISTIC MEASUREMENT SYSTEM, AND URINE ELECTRICAL CHARACTERISTIC MEASUREMENT METHOD

(71) Applicant: TERUMO KABUSHIKI KAISHA, Tokyo (JP)

(72) Inventors: Satoshi SAWADA, Hadano-shi (JP); Ken YOSHINAGA, Ayase-shi (JP)

(73) Assignee: TERUMO KABUSHIKI KAISHA, Tokyo (JP)

(21) Appl. No.: 19/702,237

(22) Filed: Jun. 9, 2026

Related U.S. Application Data
(63) Continuation of application No. PCT/JP2024/045573, filed on Dec. 24, 2024.

(30) Foreign Application Priority Data
Dec. 25, 2023 (JP) 2023-218225

(51) Int. Cl. A61B 5/20; A61M 25/00; G01N 33/487; G01N 33/493

(52) U.S. Cl. CPC A61B 5/208; A61M 25/0017; G01N 33/48707; G01N 33/493; A61M 2205/3317

(57) ABSTRACT

A urine electrical characteristic measurement device includes a drip tube that drops urine, a retainer that temporarily retains the dropped urine, and a measurement unit that measures electrical characteristics of the urine retained in the retainer.

### CROSS-REFERENCES TO RELATED APPLICATIONS

This application is a continuation of International Application No. PCT/JP2024/045573 filed on Dec. 24, 2024, which claims priority to Japanese Application No. 2023-218225 filed on Dec. 25, 2023, the entire content of both of which is incorporated herein by reference.

### TECHNOLOGICAL FIELD

The present disclosure generally relates to a urine electrical characteristic measurement device, a urine electrical characteristic measurement system, and a urine electrical characteristic measurement method.

### BACKGROUND DISCUSSION

For a patient who cannot excrete urine from the body due to illness or surgery, a urinary catheter is inserted from the urethra into the bladder, thereby urine excreted by the urinary catheter can be stored in a urinary drainage bag. A condition of the patient can be confirmed by inspecting components of the excreted urine. For example, it is known that electrical characteristics (conductivity and resistivity) of urine correlate with a urine osmotic pressure (chloride ions), and it is useful to grasp the electrical characteristics of urine in determination of a renal function and screening for diseases.

Japanese Patent Application Publication No. 2012-32353 A discloses a urine test device including a measurement unit, a transport unit, and an information processing unit, in which the transport unit includes a transport path, a sample rack holding a plurality of sample containers is transported on the transport path, and a sample is sucked from each of the sample containers by a nozzle provided in the measurement unit to measure the sample.

However, in a case where the urine test device as disclosed in Japanese Patent Application Publication No. 2012-32353 A is used for a patient who cannot excrete urine from the body, it is necessary to collect urine stored in a urinary drainage bag and store the urine in a sample container, and urine sampling is required each time a test is performed. Thus, electrical characteristics of urine excreted from the patient's body cannot be continuously measured. In addition, even if the urine stored in the urinary drainage bag is collected, it is not suitable for measuring a temporal change in electrical characteristics of fresh urine.

### SUMMARY

A urine electrical characteristic measurement device, a urine electrical characteristic measurement system, and a urine electrical characteristic measurement method capable of continuously measuring electrical characteristics of urine are disclosed.
(1) A urine electrical characteristic measurement device according to the present disclosure includes a dropper portion that drops urine, a retention portion that temporarily retains the dropped urine, and a measurement portion that measures electrical characteristics of the urine retained in the retention portion.

Here, in an embodiment of the present disclosure, (2) the urine electrical characteristic measurement device of (1) includes a plurality of first electrodes that is in continuous contact with the urine retained in the retention portion.
(3) The urine electrical characteristic measurement device of (2) includes a plurality of second electrodes that respectively abuts on the plurality of first electrodes, and the measurement portion measures electrical characteristics of urine by energizing each of the second electrodes.
(4) The urine electrical characteristic measurement device of (3) includes a spring member for reinforcing abutment between the first electrodes and the second electrodes.
(5) In the urine electrical characteristic measurement device according to any one of (1) to (4), the retention portion includes a cylindrical body, and a porous body that is disposed in the cylindrical body and temporarily retains urine.
(6) In the urine electrical characteristic measurement device of (5), some or all of the plurality of first electrodes are embedded and disposed in an outer peripheral portion of the porous body, and the cylindrical body holds the porous body and liquid-tightly fixes the plurality of first electrodes.
(7) In the urine electrical characteristic measurement device according to any one of (2) to (6), the retention portion includes a cylindrical body, and a disk-shaped first restriction portion that is disposed in the cylindrical body and restricts permeation of urine, and the plurality of first electrodes is disposed above the first restriction portion.
(8) In the urine electrical characteristic measurement device of (7), a flow path is formed in the first restriction portion, and the measurement portion measures electrical characteristics of urine retained in a space above the first restriction portion.
(9) In the urine electrical characteristic measurement device of (7) or (8), the retention portion includes a disk-shaped second restriction portion that is disposed to face the first restriction portion across the plurality of first electrodes and restricts permeation of urine.
(10) In the urine electrical characteristic measurement device of (9), a flow path is formed in a first peripheral portion of the first restriction portion, and a flow path is formed in a second peripheral portion of the second restriction portion facing the first peripheral portion across a central axis of the cylindrical body.
(11) In the urine electrical characteristic measurement device of (9) or (10), the measurement portion measures electrical characteristics of urine retained in a space between the first restriction portion and the second restriction portion.
(12) The urine electrical characteristic measurement device according to any one of (1) to (11) includes a detection portion that detects the number of drops of dropped urine, and a calculation portion that calculates a flow rate of urine based on the number of drops detected by the detection portion.
(13) A urine electrical characteristic measurement system according to the present disclosure includes the above-described urine electrical characteristic measurement device, and a storage device that stores the electrical characteristics of urine measured by the electrical characteristic measurement device.
(14) A urine electrical characteristic measurement system according to the present disclosure includes the above-described urine electrical characteristic measurement device, and a display device that displays the electrical characteristics of urine measured by the electrical characteristic measurement device.
(15) A urine electrical characteristic measurement system according to the present disclosure includes the above-described urine electrical characteristic measurement device, and a conversion device that converts the electrical characteristics of urine measured by the electrical characteristic measurement device into at least one of a urine osmotic pressure, a urine sodium chloride concentration, or a urine sodium excretion rate.
(16) A urine electrical characteristic measurement system according to the present disclosure includes the above-described urine electrical characteristic measurement device, and a determination device that determines an effect of a diuretic drug based on the electrical characteristics of urine measured by the electrical characteristic measurement device.
(17) A urine electrical characteristic measurement system according to the present disclosure includes the above-described urine electrical characteristic measurement device, and a measurement device that measures a urine oxygen partial pressure, in which the measurement device includes a correction portion that corrects the measured urine oxygen partial pressure based on the electrical characteristics of urine measured by the electrical characteristic measurement device.
(18) A urinary drainage system comprising: a urinary catheter having a balloon-shaped fixing portion configured to fix a distal portion of the urinary catheter to a bladder a patient; a urine electrical characteristic measurement device connected to a proximal end of the urinary catheter via a tube, the urine electrical characteristic device including a drip tube configured to drop urine received from the urinary catheter, a retainer configured to temporarily retain the dropped urine, a measurement unit configured to measure electrical characteristics of the urine retained in the retainer, and a plurality of first electrodes that is in continuous contact with the urine retained in the retainer; and a urine bag connected to the urine electrical characteristic measurement device
(19) A urine electrical characteristic measurement method according to the present disclosure includes temporarily retaining urine dropped from a dropper portion by a retention portion and measuring electrical characteristics of the retained urine by a measurement portion.

According to the present disclosure, it is possible to continuously measure electrical characteristics of urine.

### BRIEF DESCRIPTION OF THE DRAWINGS

FIG. 1 is a view illustrating an example of an appearance of a main part of a urine electrical characteristic measurement device of the present embodiment;

FIG. 2 is a view illustrating a first example of a configuration of the urine electrical characteristic measurement device;

FIG. 3 is a view illustrating an example of a configuration of a measurement unit;

FIG. 4 is a view illustrating a second example of the configuration of the urine electrical characteristic measurement device;

FIG. 5 is a view illustrating a third example of the configuration of the urine electrical characteristic measurement device;

FIG. 6 is a view illustrating a fourth example of the configuration of the urine electrical characteristic measurement device;

FIG. 7 is a view illustrating a fifth example of the configuration of the urine electrical characteristic measurement device;

FIG. 8 is a view illustrating an example of a configuration of a urinary drainage system of the present embodiment;

FIG. 9 is a view illustrating an example of a configuration of a urine electrical characteristic measurement system of the present embodiment;

FIG. 10 is a view schematically indicating a relationship between a urine conductivity and a urine osmotic pressure;

FIG. 11 is a view illustrating an example of a measurement result to be displayed on a display device;

FIG. 12 is a view schematically indicating a relationship between a urine conductivity and a urine sodium chloride concentration;

FIG. 13 is a view indicating a calculation formula of a urine sodium excretion rate;

FIG. 14 is a view illustrating an example of correction of a urine oxygen partial pressure; and

FIG. 15 is a view indicating an example of measurement of effects of a diuretic drug.

### DETAILED DESCRIPTION

Set forth below with reference to the accompanying drawings is a detailed description of embodiments of a urine electrical characteristic measurement device, a urine electrical characteristic measurement system, and a urine electrical characteristic measurement method

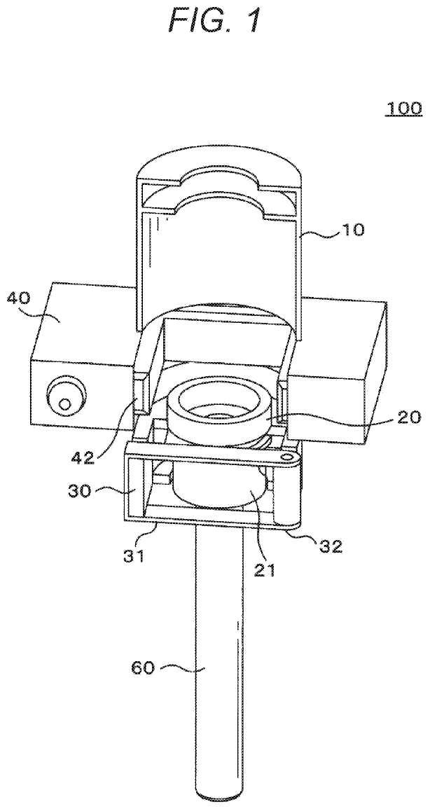

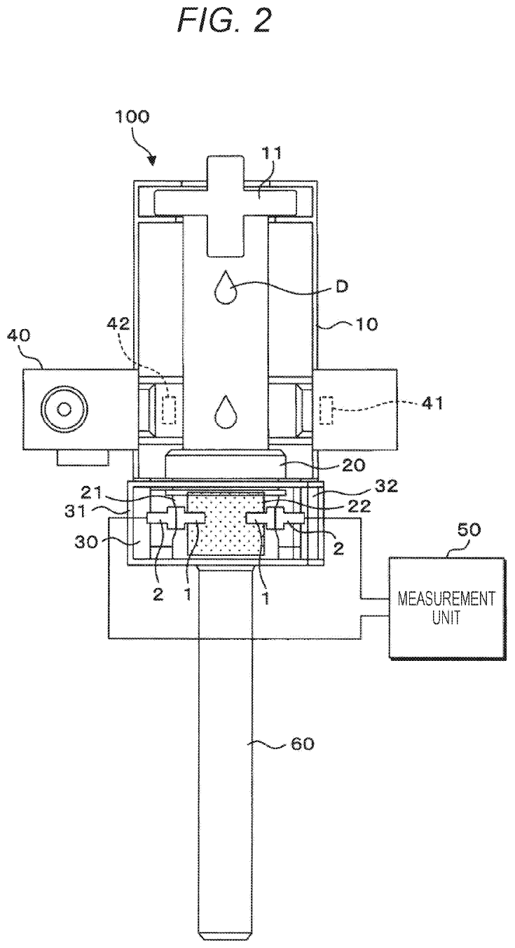

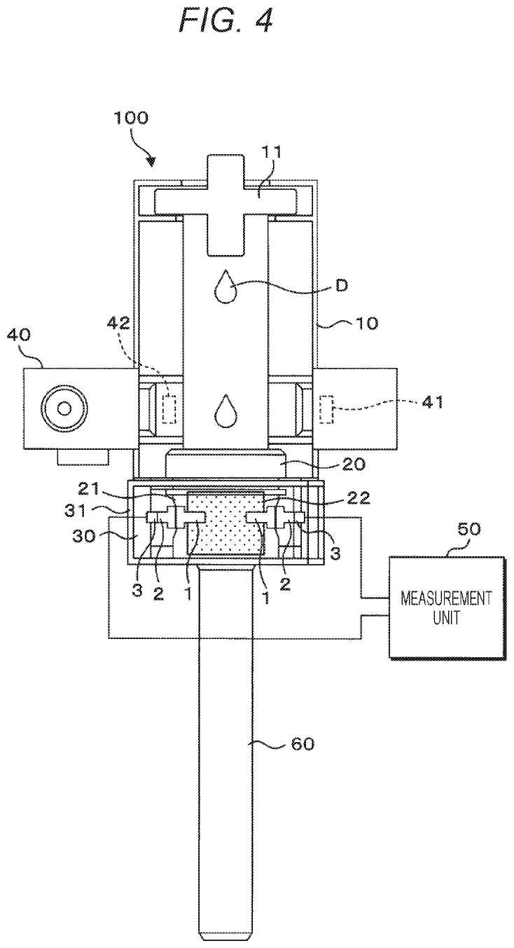

An embodiment of the present disclosure will be described below. FIG. 1 is a view illustrating an example of appearance of a main part of a urine electrical characteristic measurement device 100 of the present embodiment, and FIG. 2 is a view illustrating a first example of a configuration of the urine electrical characteristic measurement device 100. The urine electrical characteristic measurement device 100 includes a dropper portion (or drip tube) 10, a retention portion (or retainer) 20, a flow rate sensor portion (or flow rate sensor) 40, and a measurement unit 50 as a measurement portion. Note that, for convenience, FIG. 2 illustrates an internal structure of the retention portion 20 so that the internal structure can be seen. This similarly applies to FIGS. 4 to 7 described later.

The dropper portion 10 forms a transparent cylindrical container body similarly to a general drip tube. A cap portion 11 is provided above the dropper portion 10. A tube can be connected to the cap portion 11. In the dropper portion 10, a drip port having a reduced diameter is formed so that urine flowing from the tube drops as a droplet D.

The flow rate sensor portion 40 includes a light emitting portion 41 and a light receiving portion 42 arranged to face each other across the dropper portion 10. The light emitting portion 41 continuously emits required light toward the light receiving portion 42. The light receiving portion 42 can receive light emitted from the light emitting portion 41. When urine drops in the dropper portion 10, the light emitted from the light emitting portion 41 is blocked by the droplet D, and the light receiving portion 42 cannot receive light. The flow rate sensor portion 40 calculates a flow rate of urine based on a light reception signal corresponding to the received light output from the light receiving portion 42. The flow rate of urine can be calculated by, for example, flow rate of urine =number of drops per unit time ×droplet volume of one drop.

In other words, the urine electrical characteristic measurement device 100 includes a detection portion that detects the number of drops of dropped urine and a calculation portion that calculates a flow rate of urine based on the number of drops detected by the detection portion.

The retention portion 20 includes a cylindrical body 21 and a porous body 22 that is disposed in the cylindrical body 21 and temporarily retains urine. The cylindrical body 21 has a cylindrical shape, and the porous body 22 can be stored in the cylindrical body. The porous body 22 has a columnar shape and can be made of, for example, a resin foam such as sponge, and which has excellent water absorption performance due to hydrophilic water quality characteristics and a fine, continuous porous structure. The droplet (urine) dropped from the dropper portion 10 is temporarily held by the porous body 22. If an amount of urine held by the porous body 22 exceeds a predetermined amount, the urine held by the porous body 22 flows to a pipeline 60 connected to the lower side of the retention portion 20.

A frame body 30 is provided on the lower side of the dropper portion 10, and a protrusion fitted into a groove formed on an outer periphery of the retention portion 20 is formed on an inner wall of the frame body 30. The frame body 30 can define an attachment position of the retention portion 20 and hold the retention portion 20. A lock portion 31, for example, having a rectangular shape is attached to the frame body 30. The lock portion 31 is rotatably provided around a shaft body 32 on one side. By fitting the lock portion 31 into the frame body 30, the retention portion 20 can be fixed to the frame body 30, and the retention portion 20 can be disposed below the dropper portion 10.

With the above-described configuration, urine flowing in from the tube connected to the cap portion 11 drops one by one at the dropper portion 10 and is temporarily held by the retention portion 20 (porous body 22), and if the amount of the held urine exceeds a predetermined amount, the urine flows into the pipeline 60. This arrangement makes it possible to implement a structure in which fresh urine collected from the patient's body via the urinary catheter can continuously flow while keeping a certain amount (that is, slowing the flow rate).

Part or the whole of each of the plurality of first electrodes 1 is embedded and disposed in an outer peripheral portion of the porous body 22. The number of the first electrodes 1 may be, for example, two or three or more. The first electrodes 1 can be arranged, for example, at equal intervals along the outer periphery of the porous body 22. The cylindrical body 21 holds the porous body 22 and liquid-tightly fixes the plurality of first electrodes 1. The plurality of first electrodes 1 is configured to be in continuous contact with urine retained in the retention portion 20 (in the example of FIG. 2, the porous body 22). Here, “continuous contact” indicates a state in which the contact is not discrete but constant.

A plurality of second electrodes 2 capable of respectively abutting on the plurality of first electrodes 1 is provided at positions respectively corresponding to the plurality of first electrodes 1. By fitting the lock portion 31 into the frame body 30, the plurality of second electrodes 2 respectively abut on the plurality of first electrodes. The plurality of second electrodes 2 is fixed to the frame body 30. The measurement unit 50 can measure electrical characteristics of urine by energizing each of the second electrodes 2. In other words, each of the plurality of second electrodes 2 is electrically connected to each of the plurality of first electrodes 1. The measurement unit 50 can measure the electrical characteristics of urine temporarily retained (held) at the retention portion 20 (porous body 22) by applying a DC or AC voltage to the second electrodes 2. The electrical characteristics of urine include a urine conductivity or a urine resistivity.

As described above, the urine electrical characteristic measurement device 100 of the present embodiment includes the dropper portion 10 that drops urine, the retention portion 20 that temporarily retains the dropped urine, and the measurement unit (measurement portion) 50 that measures the electrical characteristics of the urine retained in the retention portion 20. The dropped urine is temporarily retained, and the electrical characteristics of the retained urine are measured, and thus, the electrical characteristics of fresh urine collected from the patient's body via the urinary catheter can be continuously measured. In addition, an air layer exists in the dropper portion 10, so that it is possible to reduce a risk that a voltage applied by the measurement unit 50 energizes the living body through a liquid. Note that hereinafter, the electrical characteristic of urine will be described as a urine conductivity. Here, “continuously measure” includes not only a case where measurement is performed literally continuously but also a case where measurement is repeatedly performed at a predetermined sampling period.

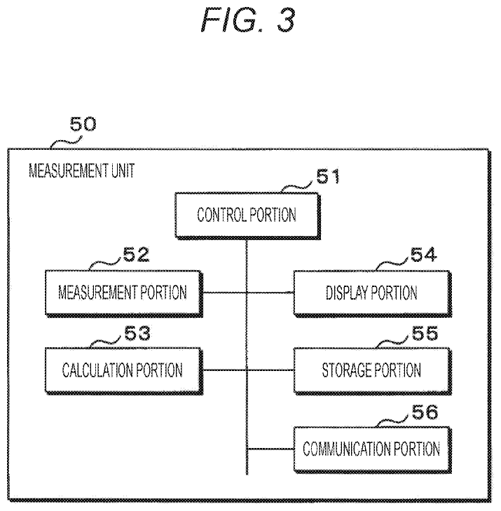

FIG. 3 is a view illustrating an example of a configuration of the measurement unit 50. The measurement unit 50 includes a control portion (or controller) 51 that controls the entire measurement unit 50. a measurement portion 52, a calculation portion 53, a display portion 54, a storage portion 55, and a communication portion 56.

The control portion 51 may be configured, for example, by incorporating a required number of central processing units (CPUs), micro-processing units (MPUs), and the like. Furthermore, the control portion 51 may be configured by combining a digital signal processor (DSP), a field-programmable gate array (FPGA), and the like. The control portion 51 can control operation of the flow rate sensor portion 40 (the light emitting portion 41, the light receiving portion 42, and the like).

The measurement portion 52 includes a DC power supply portion or an AC power supply portion and measures a conductivity of urine temporarily retained in the retention portion 20 by applying a DC or AC voltage to the plurality of second electrodes 2.

The calculation portion 53 can perform required calculation based on the measured urine conductivity. For example, the calculation portion 53 calculates a urine osmotic pressure and a urine sodium chloride concentration from the urine conductivity. Details of the calculation to be performed by the calculation portion 53 will be described later.

The display portion 54 can be configured by a liquid crystal panel, an organic electro luminescence (EL) display. An external display device may be provided instead of the display portion 54.

The storage portion 55 can be constituted by a semiconductor memory, or the like, and stores time-series data of the urine conductivity measured by the measurement portion 52, required information, and the like.

The communication portion 56 includes a communication module and can transmit and receive information to and from an external device via a communication network such as an in-hospital network.

FIG. 4 is a view illustrating a second example of the configuration of the urine electrical characteristic measurement device 100. A difference from the first example illustrated in FIG. 2 is that spring members (or elastic members) 3 are provided. The spring members 3 are provided to respectively correspond to the second electrodes 2. Each of the spring members 3 has one end fixed to the frame body 30 and the other end fixed to the second electrode 2. The spring members 3 are members for reinforcing contact between the first electrodes 1 and the second electrodes. By fitting the lock portion 31 into the frame body 30, the spring members 3 press the second electrodes 2, and the pressed second electrodes 2 press the first electrodes 1, so that contact between the first electrodes 1 and the second electrodes 2 can be ensured. The measurement unit 50 can measure electrical characteristics of urine by energizing each of the second electrodes 2. In other words, each of the plurality of second electrodes 2 is electrically connected to each of the plurality of first electrodes 1. Alternatively, the measurement unit 50 may measure the electrical characteristics of urine by energizing each of the second electrodes 2 via the spring member 3.

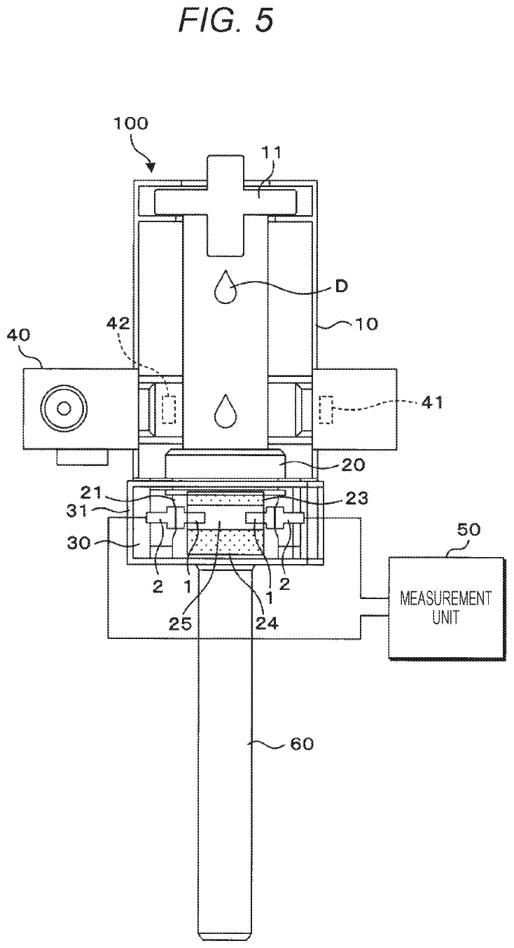

FIG. 5 is a view illustrating a third example of the configuration of the urine electrical characteristic measurement device 100. A difference from the first example illustrated in FIG. 2 is that instead of the porous body 22, two porous bodies 23, 24 are arranged to face each other while being separated from each other. As illustrated in FIG. 5, a cylindrical porous body 23 is provided on the upper side of the inside of the cylindrical body 21 having a cylindrical shape, and a cylindrical porous body 24 is provided on the lower side of the inside of the cylindrical body 21 while being separated from the porous body 23. Similarly to the first example, the porous bodies 23 and 24 can be formed with sponge.

If pore diameters of the porous bodies 23, 24 become fine (or relatively small), urine hardly permeates, and the porous bodies function as a restricting member that restricts permeation of urine. In the example of FIG. 5, the retention portion 20 includes the cylindrical body 21 and the porous body 24 as a disk-shaped first restriction portion that is disposed in the cylindrical body 21 and restricts permeation of urine, and the plurality of first electrodes 1 is disposed above the porous body 24 (first restriction portion). The retention portion 20 includes the porous body 23 as a disk-shaped second restriction portion that is disposed to face the porous body 24 across the plurality of first electrodes 1 and restricts permeation of urine. The dropped urine is once held by the porous body 23, and the urine exceeding a certain amount is retained in the space 25 between the porous body 23 and the porous body 24 and is once held by the porous body 24. If the urine held by the porous body 24 exceeds a certain amount, the urine flows from the porous body 24 to the pipeline 60.

With the above-described configuration, the measurement unit 50 can measure the conductivity of urine retained in the space 25 between the porous body 24 and the porous body 23. In addition, the configuration in which the first electrodes 1 are embedded in the porous body such as sponge is not adopted, so that it is possible to eliminate influence of a change in the retention amount of urine caused by a compression rate of the sponge, or the like, influence of pores (spaces) generated around the first electrodes 1, and it is possible to measure the urine conductivity more accurately.

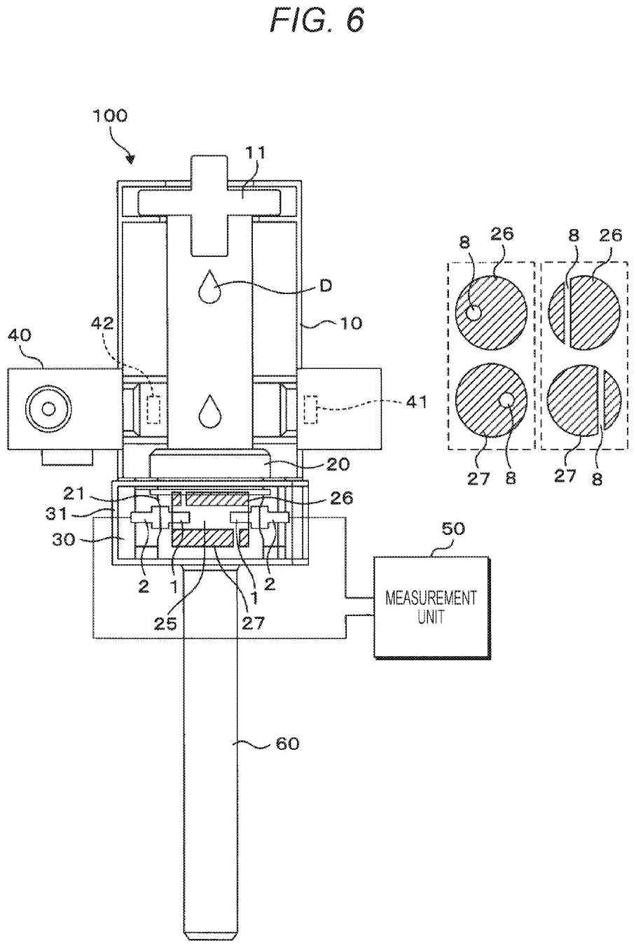

FIG. 6 is a view illustrating a fourth example of the configuration of the urine electrical characteristic measurement device 100. A difference from the third example illustrated in FIG. 5 is that plates 26, 27 are provided instead of the porous bodies 23, 24. The plates 26, 27 function as the second restriction portion and the first restriction portion, respectively, and can be formed with a film, or the like, which does not allow permeation of urine. As illustrated in FIG. 6, a hole as the flow path 8 is formed in a first peripheral portion (in the example of FIG. 6, a right peripheral portion) of the lower plate 27, and a hole as the flow path 8 is formed in a second peripheral portion (in the example of FIG. 6, a left peripheral portion) of the upper plate 26. The hole may have a circular shape when the plates 26, 27 are viewed from the center axis direction of the cylindrical body 21. Further, elongated holes may be formed in the plates 26, 27 instead of the holes. Here, the elongated holes are holes having an elongated shape when the plates 26, 27 are viewed from the center axis direction of the cylindrical body 21. As a result, a space 25 between the plate 26 and the plate 27 can be used as a urine flow path. The above is an example, and it is only necessary that the electrodes are brought into contact with a liquid while the flow of urine is restricted, and the shape of this hole may be designed in any manner in an optimum combination.

As described above, the flow path 8 is formed in the first peripheral portion of the plate 27 (first restriction portion), and the flow path 8 is formed in the second peripheral portion of the plate 26 (second restriction portion) facing the first peripheral portion across the central axis of the cylindrical body 21. The measurement unit 50 can measure the electrical characteristics of urine retained in the space 25 between the plate 27 and the plate 26. As a result, the first electrode 1 can be reliably brought into contact with urine temporarily retained in the space 25 between the plate 26 and the plate 27.

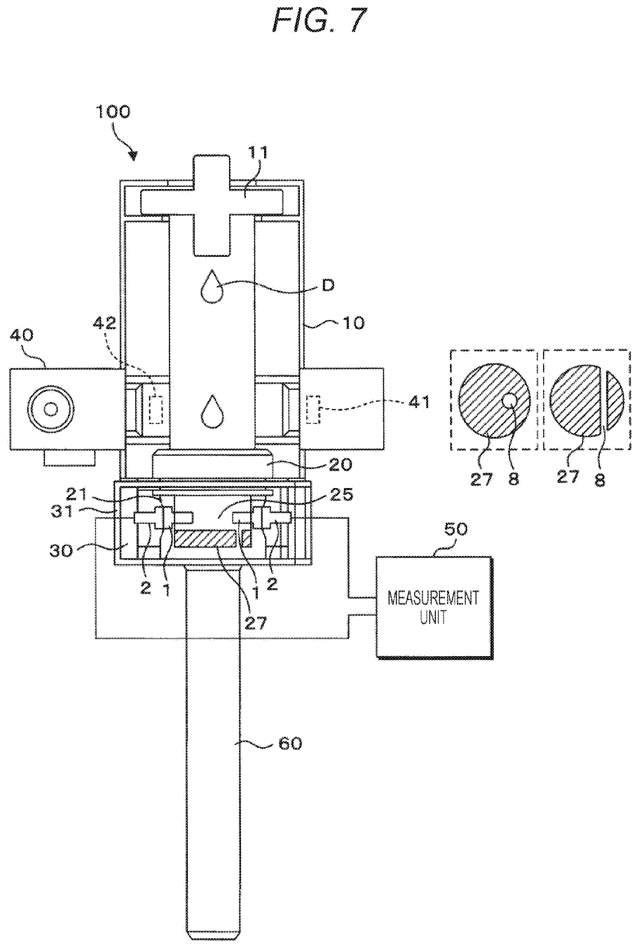

FIG. 7 is a view illustrating a fifth example of the configuration of the urine electrical characteristic measurement device 100. A difference from the fourth example illustrated in FIG. 6 is that the plate 26 (second restriction portion) is not provided. As illustrated in FIG. 7, in the fifth example, the flow path 8 is formed in the plate 27 (first restriction portion), and the measurement unit 50 can measure the electrical characteristics of urine retained in the space 25 on the plate 27. As a result, the first electrode 1 can be reliably brought into contact with urine temporarily retained in the space 25 on the plate 27.

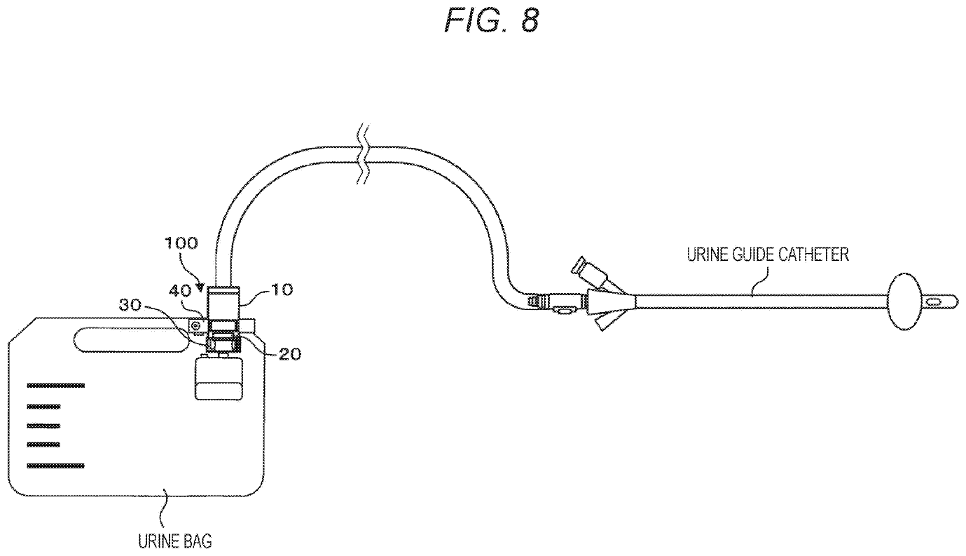

FIG. 8 is a view illustrating an example of a configuration of a urinary drainage system of the present embodiment. The urinary drainage system includes the urine electrical characteristic measurement device 100, a urinary catheter, and a urine bag. The urine bag is connected to the urine electrical characteristic measurement device 100 via the pipeline 60. In addition, the urinary catheter is connected to the tube connected to the urine electrical characteristic measurement device. A distal end of the urinary catheter is provided with a fluorescent oxygen partial pressure sensor, a temperature sensor, and a balloon-shaped fixing portion for fixing a distal portion of the urinary catheter to the bladder. According to such a configuration, urine in the body of the patient can be taken out by the urinary catheter and guided to the urine bag, and at the same time, the urine oxygen partial pressure, the temperature, and the electrical characteristics of urine can be measured.

As described above, the urine electrical characteristic measurement device 100 can measure the conductivity of urine temporarily retained in the retention portion 20. The urine electrical characteristic measurement device 100 can calculate a urine osmotic pressure from the measured urine conductivity. The urine electrical characteristic measurement device 100 can calculate a flow rate of urine.

A measurement portion that is disposed at a distal end of a fluorescent oxygen partial pressure sensor provided at a distal end of the urinary catheter and includes a light transmissive substrate and a phosphor applied on substantially the entire one surface of the substrate, is provided. The phosphor is irradiated with excitation light from the light emitting portion 131. The phosphor is excited by absorbing energy of the excitation light and emits fluorescence when a state of the phosphor returns from the excited state to the original stable state (ground state). If there is oxygen in urine, fluorescence characteristics (fluorescence intensity or phase) change due to a quenching effect. The change in fluorescence characteristics according to an oxygen concentration can be measured and converted into an oxygen concentration and a partial pressure.

The calculation portion 53 of the urine electrical characteristic measurement device 100 can calculate pO2 (oxygen partial pressure) based on a value measured by the fluorescent oxygen partial pressure sensor.

As described above, the urine electrical characteristic measurement device 100 can continuously measure the electrical conductivity of urine of the patient collected by the urinary catheter and can acquire time-series data of the urine conductivity. In addition, the urine electrical characteristic measurement device 100 can calculate time-series data of the urine osmotic pressure from the measured urine conductivity and calculate a flow rate of urine.

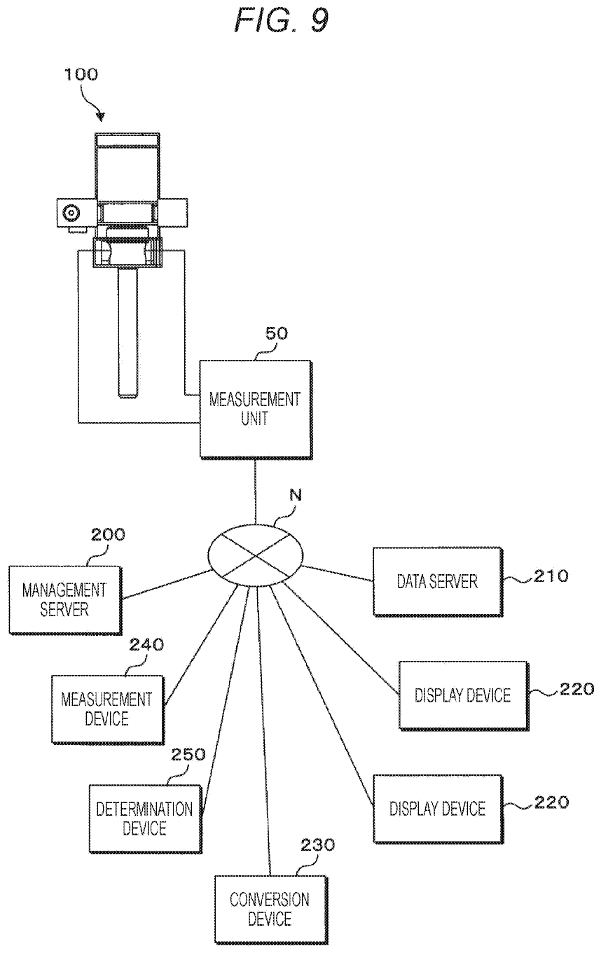

FIG. 9 is a view illustrating an example of a configuration of a urine electrical characteristic measurement system of the present embodiment. The urine electrical characteristic measurement system includes the urine electrical characteristic measurement device 100 including the measurement unit 50, a management server 200, a data server 210, a display device 220, a conversion device 230, a measurement device 240, and a determination device 250, which are connected to each other via an in-hospital network N.

The data server 210 includes a storage device and stores the urine conductivity measured by the urine electrical characteristic measurement device 100. The urine conductivity can be stored as time-series data for each patient.

The display device 220 is a device to be used by a medical worker and displays the urine conductivity measured by the urine electrical characteristic measurement device 100. The urine conductivity can be displayed as time-series data for each patient. The urine electrical characteristic measurement system may include a plurality of display devices 200.

The conversion device 230 converts the urine conductivity measured by the urine electrical characteristic measurement device 100 into at least one of a urine osmotic pressure, a urine sodium chloride concentration, or a urine sodium excretion rate.

The determination device 250 determines effects of a diuretic drug based on the urine conductivity measured by the urine electrical characteristic measurement device 100.

The measurement device 240 calculates a urine oxygen partial pressure based on the value measured by the urinary catheter. In addition, the measurement device 240 includes a correction portion that corrects the calculated urine oxygen partial pressure based on the urine conductivity measured by the urine electrical characteristic measurement device 100.

The management server 200 manages operation and information exchange of the urine electrical characteristic measurement device 100, the data server 210, the display device 220, the conversion device 230, the measurement device 240, and the determination device 250. All or some of the functions of the conversion device 230, the measurement device 240, and the determination device 250 may be integrated into the calculation portion 53 in the measurement unit 50, and the functions of the data server 210 and the display device 220 may be integrated into the storage portion 55 and the display portion 54 in the measurement unit 50. In addition, all or some of the management server 200, the data server 210, the display device 220, the conversion device 230, the measurement device 240, and the determination device 250 may be integrated into one device.

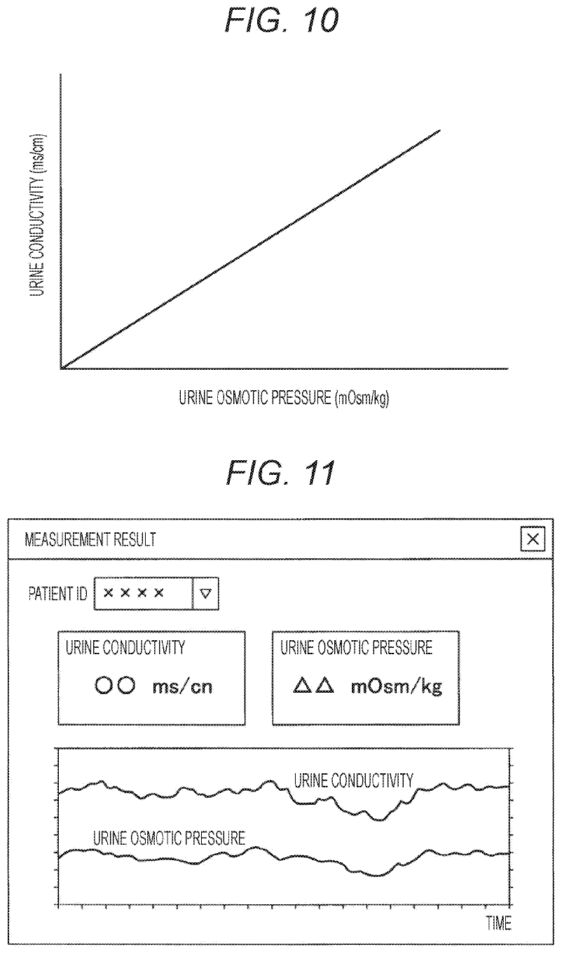

FIG. 10 is a view schematically indicating a relationship between the urine conductivity and the urine osmotic pressure. As indicated in FIG. 10, there is a correlation between the urine conductivity and the urine osmotic pressure, and as the urine osmotic pressure increases, the urine conductivity also increases in proportion to the urine osmotic pressure. Low osmotic pressure urine having a low urine osmotic pressure and high osmotic pressure urine having a high urine osmotic pressure are observed in various diseases, and there is a correlation between the urine osmotic pressure and the urine conductivity. Thus, measuring the urine conductivity of a patient is one of the means for determining the presence or absence of a disease.

FIG. 11 is a view illustrating an example of a measurement result to be displayed on the display device 220. A patient ID field for selecting a patient is displayed on a measurement result screen. By selecting or inputting the patient ID, the measurement result regarding the urine of the patient can be displayed. In the example of FIG. 11, the urine conductivity and the urine osmotic pressure of the patient are displayed as numerical values from moment to moment in real time. In addition, it is possible to display time-series data of the urine conductivity and the urine osmotic pressure acquired during a predetermined period dating back from the current time point, which makes it possible to grasp a temporal change in urine conductivity or urine osmotic pressure. Note that the data to be displayed on the measurement result screen is not limited to the urine conductivity and the urine osmotic pressure, and time-series data of the urine sodium chloride concentration, the urine sodium excretion rate, the urine oxygen partial pressure, and the like, may be displayed alone or in combination.

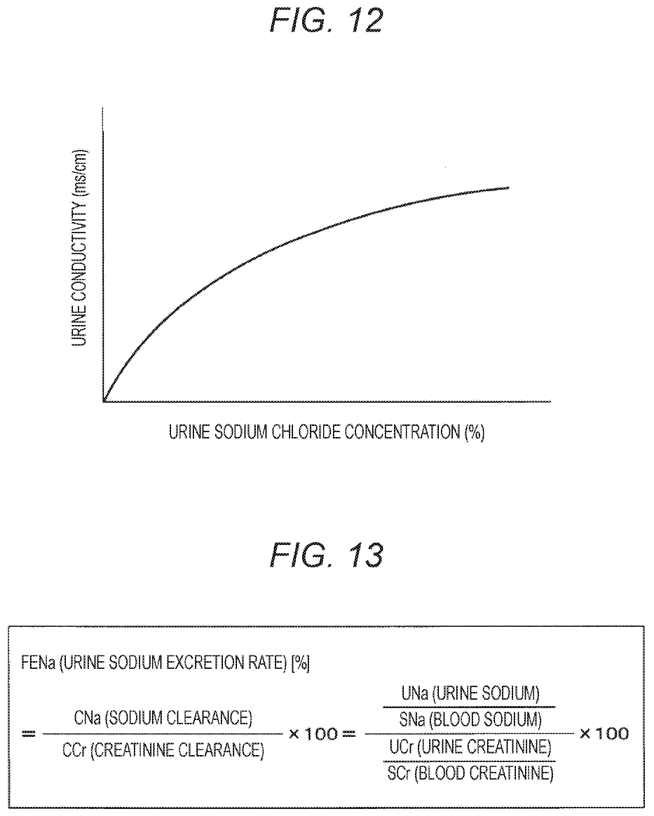

FIG. 12 is a view schematically indicating a relationship between the urine conductivity and the urine sodium chloride concentration. The urine conductivity is proportional to an electrolytic mass in an aqueous solution, and sodium chloride is the most common electrolyte in urine, and thus, there is a correlation between the urine conductivity and the urine sodium chloride concentration as schematically indicated in FIG. 12. The urine conductivity measured by the urine electrical characteristic measurement device 100 of the present embodiment accurately reproduces the correlation as indicated in FIG. 12.

FIG. 13 is a view indicating a calculation formula of the urine sodium excretion rate. The urine sodium excretion rate (FENa) is a ratio of sodium clearance CNa (excretion ability of sodium) to creatinine clearance CCr (excretion ability of creatinine), and specifically, is a value obtained by dividing the ratio of urine sodium UNa to blood sodium SNa by the ratio of urine creatinine UCr to blood creatinine SCr. A case where FENa is less than 1% is indicative of pre-renal failure, and a case where FENa exceeds 1% is indicative of renal failure. As described above, FENa can be used to distinguish between pre-renal and renal acute kidney injuries (AKI).

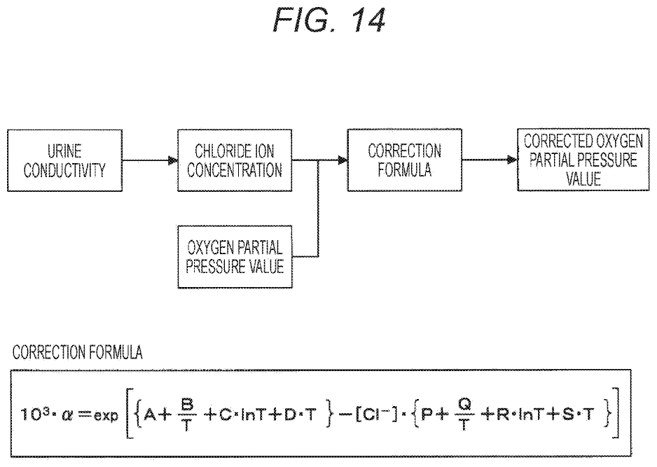

FIG. 14 is a view illustrating an example of correction of the urine oxygen partial pressure. The conversion device 230 converts the urine conductivity measured by the urine electrical characteristic measurement device 100 into a chloride ion concentration (sodium chloride concentration). The measurement device 240 calculates an oxygen partial pressure value of urine from a value measured by the urinary catheter and acquires a chloride ion concentration from the conversion device 230. The measurement device 240 corrects the calculated oxygen partial pressure value by substituting the value of the chloride ion concentration into the correction formula held in the correction portion. In the correction formula indicated in FIG. 14, T represents a temperature, and A to D and P to S are constants. A=−7.424, B=4.417×103, C=−2.927, D=4.238×10−2, P=−1.288×10−1, Q=5.344×10, R=−4.442×10−2, and S=7.145×10−4. [Cl−] is the chloride ion concentration.

If the chloride ion concentration is high due to the salting-out effect, solubility of oxygen decreases, so that the oxygen partial pressure is calculated to be higher than the actual oxygen partial pressure, and thus, the oxygen partial pressure needs to be corrected. With the above-described configuration, an accurate oxygen partial pressure can be obtained.

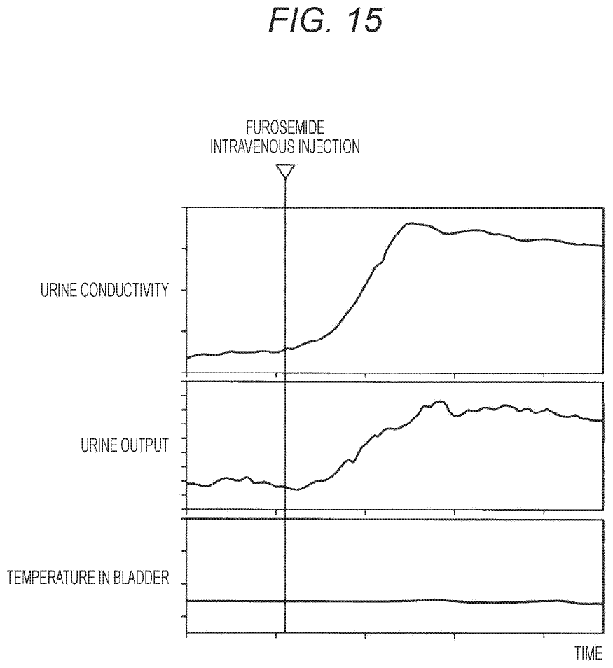

FIG. 15 is a view indicating an example of measurement of the effects of a diuretic drug. The diuretic drug (furosemide Na) most used in clinical practice has a function of increasing the urine osmotic pressure and increasing the urine output by suppressing reabsorption of sodium chloride from the primary urine. Urinary sodium chloride increases when diuresis increases by a diuretic drug, so that it is possible to determine whether a reaction in which the urine output increases after administration of furosemide is an effect by administration of furosemide by observing urine sodium chloride. The urine electrical characteristic measurement device 100 of the present embodiment can continuously measure the urine conductivity, so that it is possible to visualize whether increase in urine output after administration of furosemide to a living body is caused by the effect of furosemide.

FIG. 15 indicates a temporal change in urine conductivity, a temporal change in urine output, and a temporal change in temperature in the bladder in a case where a diuretic drug is administered to a pig. It can be seen that the urine conductivity and the urine output increase with respect to a baseline (before administration of furosemide) in 1 minute to 2 minutes after administration of furosemide. It can also be understood that the effect of furosemide is weakened after a lapse of a certain period of time. A certain threshold may be provided to determine the effect of the diuretic drug, or the effect of the diuretic drug may be determined based on a rate of change from the baseline (before administration of furosemide).

In clinical practice, a test may be performed to confirm responsiveness to furosemide, called furosemide stress test (furosemide challenge). In the furosemide stress test, a certain amount of furosemide is administered, and responsiveness is determined based on the urine output obtained after a lapse of a certain period of time (for example, 2 hours). The urine electrical characteristic measurement device 100 of the present embodiment may be used when the furosemide stress test is performed. In this case, a menu useful for performing the furosemide stress test may be displayed on the display device 220 on the display portion 54 of the measurement unit 50.

Further, whether natriuresis is performed is also useful for prognosis of acute heart failure and can be determined by an osmotic pressure (concentration). Thus, also in prognosis of acute heart failure, it is useful to continuously measure the urine conductivity by the urine electrical characteristic measurement device 100 of the present embodiment. In addition, the parameters measurable so far are useful for estimating a state of the kidney from a plurality of viewpoints by being used in combination.

The detailed description above describes embodiments of a urine electrical characteristic measurement device, a urine electrical characteristic measurement system, and a urine electrical characteristic measurement method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A urine electrical characteristic measurement device comprising:
a drip tube configured to drop urine;
a retainer configured to temporarily retain the dropped urine; and
a measurement unit configured to measure electrical characteristics of the urine retained in the retainer.

2. The urine electrical characteristic measurement device according to claim 1, comprising
a plurality of first electrodes that is in continuous contact with the urine retained in the retainer.

3. The urine electrical characteristic measurement device according to claim 2, comprising
a plurality of second electrodes that respectively abuts on the plurality of first electrodes,
wherein the measurement unit is configured to measure electrical characteristics of the urine by energizing each of the plurality of second electrodes.

4. The urine electrical characteristic measurement device according to claim 3, comprising
a spring member for reinforcing abutment between the plurality of first electrodes and the plurality of second electrodes.

5. The urine electrical characteristic measurement device according to claim 2, wherein the retainer includes:
a cylindrical body; and
a porous body that is disposed in the cylindrical body and configured to temporarily retain urine.

6. The urine electrical characteristic measurement device according to claim 5, wherein
one or more of the plurality of first electrodes are embedded and disposed in an outer peripheral portion of the porous body, and
the cylindrical body holds the porous body and liquid-tightly fixes the plurality of first electrodes.

7. The urine electrical characteristic measurement device according to claim 2, wherein the retainer includes:
a cylindrical body; and
a disk-shaped first restriction portion that is disposed in the cylindrical body and configured to restrict permeation of urine, and
the plurality of first electrodes is disposed above the disk-shaped first restriction portion.

8. The urine electrical characteristic measurement device according to claim 7, wherein
a flow path is formed in the disk-shaped first restriction portion, and
the measurement unit measures electrical characteristics of urine retained in a space above the disk-shaped first restriction portion.

9. The urine electrical characteristic measurement device according to claim 7, wherein the retainer includes
a disk-shaped second restriction portion that is disposed to face the disk-shaped first restriction portion across the plurality of first electrodes and configured to restrict permeation of urine.

10. The urine electrical characteristic measurement device according to claim 9, wherein
a flow path is formed in a first peripheral portion of the disk-shaped first restriction portion, and
a flow path is formed in a second peripheral portion of the disk-shaped second restriction portion facing the first peripheral portion across a central axis of the cylindrical body.

11. The urine electrical characteristic measurement device according to claim 9, wherein the measurement unit is configured to measure electrical characteristics of urine retained in a space between the disk-shaped first restriction portion and the disk-shaped second restriction portion.

12. The urine electrical characteristic measurement device according to claim 1, further comprising:
a detector configured to detect a number of drops of dropped urine; and
a processor configured to calculate a flow rate of urine based on the number of drops detected by the detector.

13. A urine electrical characteristic measurement system comprising:
the urine electrical characteristic measurement device according to claim 1; and
a storage device configured to store the electrical characteristics of urine measured by the electrical characteristic measurement device.

14. A urine electrical characteristic measurement system comprising:
the urine electrical characteristic measurement device according to claim 1; and
a display device configured to display the electrical characteristics of urine measured by the electrical characteristic measurement device.

15. A urine electrical characteristic measurement system comprising:
the urine electrical characteristic measurement device according to claim 1; and
a processor configured to convert the electrical characteristics of urine measured by the electrical characteristic measurement device into at least one of a urine osmotic pressure, a urine sodium chloride concentration, or a urine sodium excretion rate.

16. A urine electrical characteristic measurement system comprising:
the urine electrical characteristic measurement device according to claim 1; and
a processor configured to determine an effect of a diuretic drug based on the electrical characteristics of urine measured by the electrical characteristic measurement device.

17. A urine electrical characteristic measurement system comprising:
the urine electrical characteristic measurement device according to claim 1; and
a measurement device configured to measure a urine oxygen partial pressure, and wherein the measurement device includes:
a processor configured to correct the measured urine oxygen partial pressure based on the electrical characteristics of urine measured by the electrical characteristic measurement device.

18. A urinary drainage system comprising:
a urinary catheter having a balloon-shaped fixing portion configured to fix a distal portion of the urinary catheter to a bladder a patient;
a urine electrical characteristic measurement device connected to a proximal end of the urinary catheter via a tube, the urine electrical characteristic device including a drip tube configured to drop urine received from the urinary catheter, a retainer configured to temporarily retain the dropped urine, a measurement unit configured to measure electrical characteristics of the urine retained in the retainer, and a plurality of first electrodes that is in continuous contact with the urine retained in the retainer; and
a urine bag connected to the urine electrical characteristic measurement device.

19. A urine electrical characteristic measurement method comprising:
temporarily retaining urine dropped from a drip tube with a retainer; and
measuring electrical characteristics of the retained urine with a measurement unit.

20. The urine electrical characteristic measurement method according to claim 19, further comprising:
continuously contacting the urine retained in the retainer with a plurality of first electrodes, the plurality of first electrodes being abutted by a plurality of second electrodes; and
measuring electrical characteristics of the urine by energizing each of the plurality of second electrodes.
